(19) United States
(12) Patent Application Publication
(10) Pub. No.: US 2026/0036503 A1
(43) Pub. Date: Feb. 5, 2026

(54) Method, device, and computing apparatus for calculating fracturing fluid leak-off volume in reservoir matrices

(71) Applicants: Li Ben; Quanjun Wang; Li Hui

(72) Inventors: Li Ben; Quanjun Wang; Li Hui

(73) Assignee: STenergy LLC, Houston, TX (US)

(21) Appl. No.: 19/231,293

(22) Filed: Jun. 6, 2025

(51) Int. Cl. G01N 3/12; G01N 33/24

(52) U.S. Cl. CPC G01N 3/12; G01N 33/24; G01N 2203/0067

(57) ABSTRACT

This utility patent discloses a method, device, and computing apparatus for calculating fracturing fluid leak-off volume in reservoir matrices, within the field of petroleum exploitation. The method includes: acquiring pressure data from measurement points in core samples during fracturing fluid damage tests; determining fluid-induced damage length based on the data; calculating permeability variation parameters as a function of invasion depth; computing fluid invasion depth using fracturing fluid density, viscosity, and permeability variation; and calculating total leak-off volume based on average fracture height and length of primary and branch fractures. The method enables fast and accurate estimation of fluid loss by incorporating key factors such as permeability variation with depth, fluid density, and viscosity, offering a reliable foundation for hydraulic fracturing design.

### TECHNICAL FIELD

This specification relates to the technical field of petroleum extraction, and in particular, to a method, apparatus, and computer device for calculating fracturing fluid loss in reservoir matrix systems.

### BACKGROUND OF THE INVENTION

In the process of reservoir hydraulic fracturing, the fluid loss of fracturing fluid is a key parameter for evaluating both the effectiveness of the fracturing fluid and the scale of fracture creation. Fluid loss directly impacts the effective volume of fracturing fluid retained within the fracture, which is defined as the difference between the total injected volume and the leak-off volume. The ratio of the retained fracturing fluid volume within the fracture to the injected volume is defined as the fracturing fluid efficiency. A higher fracturing fluid efficiency indicates greater effectiveness in fracture propagation. Accurate evaluation of fluid loss enables precise design of fracturing fluid volumes to ensure that the treatment volume aligns with the targeted stimulated reservoir volume.

The fluid loss of the fracturing fluid also affects the internal fracture pressure. Under constant injection rates, greater fluid loss results in lower fracture pressure, leading to reduced fracture width. A narrower fracture increases the resistance to proppant transport and may hinder proppant placement. Inadequate proppant distribution ultimately limits fracture conductivity and impairs the reservoir's capacity to achieve efficient hydrocarbon production.

Fracturing fluid loss into the reservoir is mainly governed by matrix permeability, porosity, and the fracture pressure gradient. Existing technologies lack a comprehensive and accurate method for calculating the volume of fracturing fluid loss into the reservoir matrix.

In view of the above, embodiments of the present disclosure are directed to providing a method, an apparatus, and a computer device for calculating the fluid loss volume of fracturing fluid within a reservoir matrix.

### SUMMARY OF THE INVENTION

To address the above-mentioned issues in the prior art, the objective of the embodiments disclosed in this specification is to provide a method, device, and computer equipment for calculating the fracturing fluid loss in the reservoir matrix. This aims to resolve the problem in the existing technology wherein there is a lack of methodology for accurately quantifying the fluid loss of fracturing fluids.

In a first aspect, the embodiments of the present disclosure provide a method for calculating the fracturing fluid loss volume within the reservoir matrix, comprising:
Acquire pressure data from each pressure measurement point along the core during the fracturing fluid-induced damage experiment conducted on the core sample;
Based on the pressure data, the fracturing fluid invasion (or damage) length of the core is determined;
Based on the fracturing fluid damage length, the variation parameter of the reservoir matrix permeability to fracturing fluid as a function of invasion depth is determined;
Based on the density and viscosity of the fracturing fluid, as well as the depth-dependent permeability variation parameter of the reservoir matrix to the fracturing fluid, the fracturing fluid loss depth within the reservoir is determined. The calculated reservoir fracturing fluid loss depth is expressed as:

$${D}_{l o s s} = \frac{90.35 \times {ρ}_{fluid} {{K}_{m} ( i )}^{1.5} ( {P}_{fracture} - {P}_{formation} )}{{μ}_{fluid}^{2} {e}^{( \frac{1.5}{b} ) ln ( {K}_{r o c k} / a )}}$$

In which: Dₗₒₛₛ is the fracturing fluid loss depth, cm; ρ_{fluid} is the density of the fracturing fluid, g/cm³; Kₘ(i) is the permeability at a position corresponding to a penetration depth of i, 10⁻³ μm²; i is the penetration depth of the fracturing fluid into the reservoir matrix, cm; P_{fracture} is the fluid pressure inside the fracture, MPa; P_{formation} is the reservoir pore pressure, MPa; μ_{fluid} is the viscosity of the fracturing fluid, mPa. S; K_{rock} is the intrinsic permeability of the reservoir matrix, 10⁻³ μm²; a, b are the dimensionless regression coefficients of the porosity-permeability relationship for the reservoir matrix;
Based on the average fracture height and fracture length of the primary hydraulic fracture, the average fracture height and fracture length of the branch fractures, and the calculated fracturing fluid loss depth within the reservoir, the total volume of fracturing fluid loss is determined.

Specifically, the determination of the fracturing fluid invasion (damage) length within the core based on the pressure data further comprises:
Obtaining pressure data from multiple pressure measurement points along the core holder during a fracturing fluid damage experiment conducted under conditions of initial inlet pressure, initial outlet pressure, and a predetermined fracturing duration;
Calculating the differential pressure gradient deviation coefficient for each pressure measurement point based on the pressure data, the initial inlet pressure, and the initial outlet pressure;
Determining whether any pressure measurement point exhibits a pressure gradient deviation coefficient that satisfies a predefined condition;
If one or more pressure measurement points meet the predefined condition, the fracturing fluid damage length is determined based on the location of these qualifying pressure measurement points and the number of repetitions of the fracturing fluid damage experiment;
If none of the pressure measurement points satisfy the predefined condition, the initial inlet pressure is updated, and the fracturing fluid damage experiment is repeated for the predetermined fracturing duration until at least one pressure measurement point satisfies the predefined pressure gradient deviation condition.

Specifically, the pressure gradient deviation coefficient is calculated using the following formula:

$${η}_{j} = ( {G}_{Pj} - {G}_{rock} ) / {G}_{rock}$$

In which: ηⱼ is the pressure gradient deviation coefficient at the j-th pressure measurement point, dimensionless; G_{Pj} is the pressure gradient at the j-th pressure sensing point, MPa/cm; G_{rock} is the pressure gradient between the outlet and the inlet of the core holder, MPa/cm;

$${G}_{Pj} = \frac{{P}_{j} - {P}_{j - 1}}{Δ L}$$

$${G}_{rock} = \frac{{P}_{i n} - {P}_{out}}{L}$$

In which: Pⱼ is the pressure measured at the j-th pressure monitoring point, MPa; Pⱼ₋₁ the pressure at the (j−1)th pressure measurement point, MPa; ΔL is the core length between two adjacent pressure measurement points, cm; Pᵢₙ is the pressure at the inlet of the core holder, MPa; Pₒᵤₜ is the pressure at the outlet end of the core holder, MPa; L is the length of the core, cm.

Further, when a predicted point exhibiting a pressure gradient deviation coefficient that meets a predetermined condition is identified, the fracturing fluid damage length is determined based on the pressure measurement points satisfying the predetermined condition of the pressure gradient deviation coefficient and the number of repetitions of the fracturing fluid damage test:

$${L}_{damage} = n L + j Δ L$$

In which: L_{damage} is the fracturing fluid invasion damage length, cm; n is the number of repetitions of the fracturing fluid damage test; L is the length of the core, cm; ΔL is the core length between two adjacent pressure measurement points, cm.

Furthermore, when the pressure measurements at all monitoring points fail to meet the predefined criteria, the initial inlet pressure is updated. Specifically, the update is performed as follows:
The initial inlet pressure is replaced by the outlet pressure of the core holder recorded at the moment the fracturing duration reaches the preset pressure holding time during the previous fracturing fluid damage test.

Specifically, based on the determined fracturing fluid damage length, the permeability variation parameter of the reservoir matrix to the fracturing fluid as a function of penetration depth is calculated as follows:

$${K}_{m} ( i ) = \{ \begin{matrix}{K}_{rock} ( 1 - {η}_{damage} + {η}_{damage} \frac{tansig ( i )}{tansig ( Ldamage )} ) & ( i \leq {L}_{damage} ) \\ {K}_{rock} & ( {L}_{damage} \leq i \leq {D}_{loss} )\end{matrix}$$

In which: Kₘ(i) is the permeability at a position corresponding to a penetration depth of i, 10⁻³ μm²; K_{rock} is the intrinsic permeability of the reservoir matrix, 10⁻³ μm²; L_{damage} is the fracturing fluid invasion damage length, cm; tan sig is the transfer function, which is specifically calculated by the following expression:

$$tansig ( i ) = \frac{{e}^{i} - {e}^{- i}}{{e}^{i} + {e}^{- i}}$$

i is the penetration depth of the fracturing fluid into the reservoir matrix, cm.

Furthermore, based on the average fracture height and fracture length of the primary hydraulic fracture, the average fracture height and fracture length of the branch fractures, and the calculated fracturing fluid loss depth within the reservoir, the total fluid loss volume of the fracturing fluid is determined as follows:

$${V}_{loss} = {D}_{loss} {e}^{( \frac{1}{b} ) l n ( {K}_{m} ( i ) / a )} ( {H}_{fmain} {L}_{fmain} + {H}_{fbranch 1} {L}_{fbranch 1} + {H}_{fbranch 2} {L}_{fbranch 2} + {H}_{fbranchn} {L}_{fbranchn} ) \times 1 {0}^{- 2}$$

In which: Vₗₒₛₛ is the total fluid loss volume of the fracturing fluid, m³; Dₗₒₛₛ is the fracturing fluid loss depth, cm; Kₘ(i) is the permeability at a position corresponding to a penetration depth of i, 10⁻³ μm²; H_{fmain} is the average fracture height of the primary hydraulic fracture, m; L_{fmain} is the half-length of primary hydraulic fracture, m; H_{fbranch1} is the average fracture height for hydraulic fracture of branch 1, m; L_{fbranch1} is the half-length for hydraulic fracture of branch 1, m; H_{fbranch2} is the average fracture height for hydraulic fracture of branch 2, m; L_{fbranch2} is the half-length for hydraulic fracture of branch 2, m; H_{fbranchn} is the average fracture height for hydraulic fracture of branch n, m; L_{fbranchn} is is the half-length for hydraulic fracture of branch n, m.

A device for calculating the fluid loss volume of fracturing fluid in a reservoir matrix is provided in a second aspect of the present specification, comprising:
An acquisition module, configured to obtain pressure data from various measurement points along the core during the laboratory simulation of fracturing fluid-induced damage;
A determination module, configured to determine the fracturing fluid invasion length in the core based on the acquired pressure data;
A permeability calculation module, configured to compute the variation parameter of the reservoir matrix permeability to fracturing fluid as a function of invasion depth, based on the determined invasion length;
A fluid loss depth calculation module, configured to calculate the fracturing fluid loss depth in the reservoir matrix based on the fracturing fluid density, fracturing fluid viscosity, and the permeability variation parameter of the reservoir matrix with respect to invasion depth, wherein the fracturing fluid loss depth is:

$${D}_{l o s s} = \frac{90.35 \times {p}_{fluid} {{K}_{m} ( i )}^{1.5} ( {P}_{fracture} - {P}_{formation} )}{{μ}_{fluid}^{2} {e}^{( \frac{1.5}{b} ) l n ( {K}_{r o c k} / a )}}$$

In which: Dₗₒₛₛ is the fracturing fluid loss depth, cm; ρ_{fluid} is the density of the fracturing fluid, g/cm³; Kₘ(i) is the permeability at a position corresponding to a penetration depth of i, 10⁻³ μm²; i is the penetration depth of the fracturing fluid into the reservoir matrix, cm; P_{fracture} is the fluid pressure inside the fracture, MPa; P_{formation} is the reservoir pore pressure, MPa; μ_{fluid} is the viscosity of the fracturing fluid, mPa·s; K_{rock} is the intrinsic permeability of the reservoir matrix, 10⁻³ μm²; a, b are the dimensionless regression coefficients of the porosity-permeability relationship for the reservoir matrix;
Total Fluid Loss Calculation Module, configured to calculate the total fracturing fluid loss based on the average fracture height and fracture length of the primary hydraulic fracture, the average fracture height and fracture length of the branch fractures, and the calculated fracturing fluid loss depth within the reservoir.

In a third aspect, the present specification provides a computer device comprising a memory, a processor, and a computer program stored in the memory and executable on the processor, wherein the processor, when executing the computer program, is configured to implement the method as described in the aforementioned technical solutions.

By employing the aforementioned technical solutions, the method, apparatus, and computer device for calculating the fracturing fluid loss volume within the reservoir matrix, as provided in the embodiments of the present specification, enable rapid and accurate estimation of fracturing fluid loss by accounting for critical factors such as reservoir permeability, fracturing fluid density, and fracturing fluid viscosity. This facilitates comprehensive analysis of fluid loss behavior during the hydraulic fracturing process and provides a reliable basis for the optimal design of fracturing fluid volume in reservoir stimulation operations.

To make the objectives, features, and advantages of the embodiments of the present specification more apparent and comprehensible, preferred embodiments are presented below and described in detail in conjunction with the accompanying drawings.

### DESCRIPTION OF THE DRAWINGS

In order to more clearly illustrate the technical solutions of the embodiments of the present disclosure or the prior art, the drawings required for the description of the embodiments or the prior art will be briefly introduced below. It should be understood that the following drawings merely represent some embodiments of the present disclosure. For those skilled in the art, other drawings may also be derived based on these figures without any inventive effort.

FIG. 1 is a schematic structural diagram of a method for calculating fracturing fluid loss in a reservoir matrix, as provided in an embodiment of the present disclosure;

FIG. 2 is a schematic flow diagram illustrating the step of determining the fracturing fluid invasion damage length of a core based on pressure data;

FIG. 3 is a schematic structural diagram of an apparatus for calculating fracturing fluid loss in a reservoir matrix, as provided in an embodiment of the present disclosure;

FIG. 4 is a schematic diagram illustrating a computer device applicable to implement the methods described in the present disclosure.

### DESCRIPTION OF REFERENCE NUMERALS

31: Acquisition module
32: Determination module
33: Permeability computation module
34: Fluid loss depth calculation module
35: Total fluid loss calculation module
402: Computer device
404: Processor
406: Memory
408: Drive mechanism
410: Input/Output module
412: Input device
414: Output device
416: Presentation device
418: Graphical User Interface (GUI)
420: Network interface
422: Communication link
424: Communication bus

### SPECIFIC EMBODIMENTS

The technical solutions of the embodiments of the present disclosure will now be clearly and comprehensively described in conjunction with the accompanying drawings. It is evident that the described embodiments constitute only a portion of the embodiments of the present disclosure, rather than the entirety. Based on the embodiments disclosed herein, all other embodiments obtainable by those skilled in the art without the exercise of inventive effort shall fall within the scope of protection of the present disclosure.

It should be noted that the terms “first,” “second,” and the like as used in the present specification, claims, and accompanying drawings are merely for the purpose of distinguishing between similar elements and are not necessarily intended to indicate a specific sequence or chronological order. It should be understood that such terms may be used interchangeably where appropriate, so that the embodiments of the present disclosure can be practiced in sequences other than those illustrated or described herein.

In addition, the terms “comprise,” “include,” “have,” and any of their derivatives are intended to be non-exclusive. For example, a process, method, apparatus, product, or system that comprises a series of steps or elements is not necessarily limited to only those explicitly enumerated, but may also include other steps or elements not expressly listed, or inherent to such process, method, apparatus, product, or system.

To solve the above problems, embodiments of the present disclosure provide a method, device, and computer equipment for calculating the fracturing fluid loss in the reservoir matrix, which can address the issue in the prior art of difficulty in rapidly and accurately calculating fluid loss.

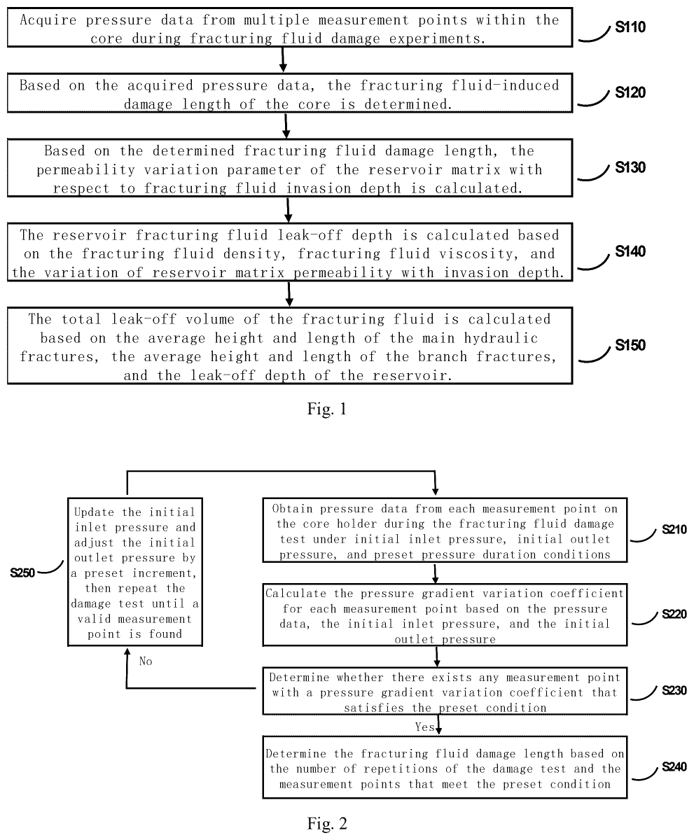

FIG. 1 is a schematic diagram illustrating the steps of a method for calculating the fracturing fluid loss in the reservoir matrix provided in an embodiment of the present disclosure. The method steps provided in the present disclosure are described as in the embodiments or flowcharts, but additional or fewer steps may be included based on conventional or non-creative efforts. The step order listed in the embodiment is only one example of execution order among many possibilities and does not represent the only execution sequence. In actual system or device implementation, the method steps may be executed sequentially or in parallel as shown in the embodiments or drawings. Specifically, as shown in FIG. 1, the method may include:

S110: Acquiring the pressure data from each pressure measurement point along the core during the fracturing fluid damage experiment of the core.

S120: Determining the fracturing fluid damage length of the core based on the acquired pressure data.

S130: Calculating the permeability variation parameter of the reservoir matrix with respect to penetration depth, based on the determined damage length.

S140: Calculating the reservoir fracturing fluid loss depth based on the fracturing fluid density, fracturing fluid viscosity, and the permeability variation parameter of the reservoir matrix with respect to penetration depth.

S150: Calculating the total volume of fracturing fluid loss based on the average fracture height and length of the main hydraulic fracture, the average fracture height and length of the branch fractures, and the calculated fracturing fluid loss depth.

The method for calculating fracturing fluid loss in the reservoir matrix provided in the embodiment of the present disclosure can take into account the effects of reservoir permeability, fracturing fluid density, and fracturing fluid viscosity, thereby enabling fast and accurate calculation of the fracturing fluid loss in the reservoir. This facilitates analysis of the fluid loss behavior during the reservoir fracturing process and provides a reliable basis for the rational design of the fracturing fluid volume.

Specifically, as shown in FIG. 2, step S120: determining the fracturing fluid damage length of the core based on the pressure data, may further include:

S210: Acquiring the pressure data from each pressure measurement point on the core holder under the conditions of initial inlet pressure, initial outlet pressure, and a preset fracturing time during the fracturing fluid damage test.

In particular, the reservoir fluid pressure P_{formation} is obtained based on reservoir logging data and formation testing data, while the fracture fluid pressure P_{fracture} is derived from the fracturing design and pumping schedule. The pressure at the inlet of the core holder is initialized using the fracture fluid pressure P_{fracture}, and the outlet pressure is initialized using the reservoir fluid pressure P_{formation}. Multiple pressure measurement points are configured and are uniformly distributed at equal intervals along the length of the core holder from the inlet end to the outlet end. By way of example, four pressure measurement points may be set, denoted as Pⱼ(j=1, 2, 3, 4). Accordingly, P₁ is positioned at the inlet of the core holder, and P₄ is positioned at the outlet.

S220: Based on the pressure data obtained from each pressure measurement point, along with the initial inlet pressure and the initial outlet pressure, the pressure gradient deviation coefficient at each measurement point is calculated.

The pressure gradient differential coefficient is determined according to the following equation:

$${η}_{i} = ( {G}_{P i} - {G}_{rock} ) / {G}_{rock}$$

In which: ηⱼ is the pressure gradient deviation coefficient at the j-th pressure measurement point, dimensionless; G_{Pj} is the pressure gradient at the j-th pressure sensing point, MPa/cm; G_{rock} is the pressure gradient between the outlet and the inlet of the core holder, MPa/cm;

In detail, the parameters are defined as follows:

$${G}_{P j} = \frac{{P}_{j} - {P}_{j - 1}}{Δ L}$$

$${G}_{rock} = \frac{{P}_{i n} - {P}_{out}}{L}$$

In which: Pⱼ is the pressure measured at the j-th pressure monitoring point, MPa; Pⱼ₋₁ the pressure at the (j−1)th pressure measurement point, MPa; ΔL is the core length between two adjacent pressure measurement points, cm; Pᵢₙ is the pressure at the inlet of the core holder, MPa; Pₒᵤₜ is the pressure at the outlet end of the core holder, MPa; L is the length of the core, cm.

S230: Determine whether there exists at least one pressure measurement point at which the pressure gradient deviation coefficient satisfies a predefined condition.

In the embodiments of this specification, the predefined condition refers to the pressure gradient deviation coefficient being less than or equal to a specified threshold. Specifically, the threshold for the pressure gradient deviation coefficient may be set to 0.1. Accordingly, it is determined whether the pressure gradient deviation coefficient ηⱼ at each pressure measurement point is less than or equal to 0.1 after the fracturing fluid damage test has been conducted for the designated fracture duration.

S240: When there exists at least one pressure measurement point whose pressure gradient deviation coefficient satisfies the predefined condition, the fracturing fluid damage length is determined based on such qualified pressure measurement point(s) and the number of repeated fracturing fluid damage experiments.

At this point, the fracturing fluid damage length is:

$${L}_{damage} = n L + j Δ L$$

In which: L_{damage} is the fracturing fluid invasion damage length, cm; n is the number of repetitions of the fracturing fluid damage test; L is the length of the core, cm; ΔL is the core length between two adjacent pressure measurement points, cm; j is the index of j-th the pressure measurement point.

It is noted that the number of repetitions n of the fracturing fluid damage experiment is defined as the total number of experimental iterations minus one. In other words, if after the first fracturing fluid damage test a pressure gradient deviation coefficient is found to satisfy the predefined criterion at a designated prediction point, then n=0, and the fracturing fluid damage length is given by L_{damage}=jΔL.

Step S250: If none of the measured pressure points meet the predefined criterion, the initial inlet pressure is updated, and the fracturing fluid damage experiment is repeated for a predefined injection duration, until at least one pressure measurement point exhibits a pressure gradient deviation coefficient satisfying the criterion.

Specifically; when all pressure gradient deviation coefficients at the measurement points are greater than 0.1, the initial inlet pressure is updated using the outlet pressure of the core holder from the previous test—this being the pressure measured at point P₄ when the injection duration reaches the predefined time. The outlet pressure of the core holder is set to the reservoir pore pressure P_{formation}, and the fracturing fluid damage experiment is repeated using the same core sample within the core holder for the predefined injection duration. At this point, the total number of experimental iterations is 2, and the number of repetitions is 1.

A determination is then made whether any pressure measurement point now satisfies the predefined criterion for the pressure gradient deviation coefficient. If not, the inlet pressure of the core holder is updated again, and the experiment is repeated until a pressure measurement point meets the criterion.

Further, step S130: Based on the determined fracturing fluid damage length L_{damage}, a parameter representing the variation of the matrix permeability to fracturing fluid with respect to penetration depth is calculated. At this point, the fracturing fluid damage length is:

$${K}_{m} ( i ) = \{ \begin{matrix}{K}_{rock} ( 1 - {η}_{damage} + {η}_{damage} \frac{tansig ( i )}{tansig ( Ldamage )} ) & ( i \leq {L}_{damage} ) \\ {K}_{rock} & ( {L}_{damage} \leq i \leq {D}_{loss} )\end{matrix}$$

In which: Kₘ(i) is the permeability at a position corresponding to a penetration depth of i, 10⁻³ μm²; K_{rock} is the intrinsic permeability of the reservoir matrix, 10⁻³ μm²; η_{damage} is the fracturing fluid damage coefficient, dimensionless; L_{damage} is the fracturing fluid invasion damage length, cm; tansig is the transfer function, which is specifically calculated by the following expression:

$$tansig ( i ) = \frac{{e}^{i} - {e}^{- i}}{{e}^{i} + {e}^{- i}}$$

i is the penetration depth of the fracturing fluid into the reservoir matrix, cm.

During the hydraulic fracturing process of a reservoir, fluid loss control additives are typically incorporated into the fracturing fluid system to reduce fluid loss and enhance the operational efficiency of the fracturing fluid. Commonly used fluid loss additives include silica flour, quartz powder, talc, marble powder, rosin, and copolymers of styrene and toluene. Due to the presence of such additives, the permeability of the reservoir matrix undergoes variation with respect to the penetration depth of the fracturing fluid.

In the embodiments described herein, the variation in penetration depth is taken into account when calculating the fracturing fluid loss depth. The intrinsic matrix permeability Kₘ of the undisturbed formation is accordingly refined to a depth-dependent permeability Kₘ(i), which varies as a function of the fluid's invasion depth. This enables the quantification of the fracturing fluid loss volume as a function of penetration depth, thereby enhancing the accuracy of fluid loss estimation.

### Furthermore, in Step S140:

Based on the fracturing fluid density, fluid viscosity, and the depth-dependent variation in reservoir matrix permeability; the fracturing fluid loss depth within the formation is computed. Specifically, the reservoir fracturing fluid loss depth is determined using the following expression:

$${D}_{l o s s} = \frac{90.35 \times {p}_{fluid} {{K}_{m} ( i )}^{1.5} ( {P}_{fracture} - {P}_{formation} )}{{μ}_{fluid}^{2} {e}^{( \frac{1.5}{b} ) l n ( {K}_{rock} / a )}} \times {10}^{2.5}$$

In which: Dₗₒₛₛ is the fracturing fluid loss depth, cm; ρ_{fluid} is the density of the fracturing fluid, g/cm³; Kₘ(i) is the permeability at a position corresponding to a penetration depth of i, 10⁻³ μm²; i is the penetration depth of the fracturing fluid into the reservoir matrix, cm; P_{fracture} is the fluid pressure inside the fracture, MPa; P_{formation} is the reservoir pore pressure, MPa; μ_{fluid} is the viscosity of the fracturing fluid, mPa·s; K_{rock} is the intrinsic permeability of the reservoir matrix, 10⁻³ μm²; a, b are the dimensionless regression coefficients of the porosity-permeability relationship for the reservoir matrix.

Finally, Step S150: based on the average fracture height and fracture length of the primary hydraulic fracture, the average fracture height and fracture length of the branch fractures, and the calculated fracturing fluid loss depth within the reservoir, the total fracturing fluid loss volume is determined, further expressed as:

$${V}_{loss} = {D}_{loss} {e}^{( \frac{1}{b} ) l n ( {K}_{m} ( i ) / a )} ( {H}_{fmain} {L}_{fmain} + {H}_{fbranch 1} {L}_{fbranch 1} + {H}_{fbranch 2} {L}_{fbranch 2} + {H}_{fbranchn} {L}_{fbranchn} ) \times 1 {0}^{- 2}$$

In which: Vₗₒₛₛ is the total fluid loss volume of the fracturing fluid, m3; Dₗₒₛₛ is the fracturing fluid loss depth, cm; Kₘ(i) is the permeability at a position corresponding to a penetration depth of i, 10⁻³ μm²; H_{fmain} is the average fracture height of the primary hydraulic fracture, m; L_{fmain} is the half-length of primary hydraulic fracture, m; H_{fbranch1} is the average fracture height for hydraulic fracture of branch 1, m; L_{fbranch1} is the half-length for hydraulic fracture of branch 1, m; H_{fbranch2} is the average fracture height for hydraulic fracture of branch 2, m; L_{fbranch2} is the half-length for hydraulic fracture of branch 2, m; H_{fbranchn} is the average fracture height for hydraulic fracture of branch n, m; L_{fbranchn} is the half-length for hydraulic fracture of branch n, m.

The total fracture surface area, denoted as A_{f}, may be defined as:

$${A}_{f} = {H}_{fmain} {L}_{fmain} + {H}_{f b ranch 1} {L}_{fbranch 1} + {H}_{fbranch 2} {L}_{fbranch 2} + … + {H}_{fbranchn} {L}_{fbranchn}$$

Accordingly, the following relationship is established:

$${V}_{l o s s} = {D}_{l o s s} {e}^{( \frac{1}{b} ) ln ( {K}_{m} ( i ) / a )} {A}_{f} \times 1 {0}^{- 2}$$

The fluid-loss area of the fracturing fluid represents another critical parameter for evaluating the total fluid loss into the reservoir formation. The fluid-loss area is defined as the cumulative surface area of all fractures in direct contact with the fracturing fluid. Microseismic monitoring results have indicated that the hydraulic fracture network in the reservoir typically exhibits a primary fracture and multiple branch fractures. The primary fracture generally exhibits the greatest fracture length and height, while the branch fractures are more complex and exist in greater numbers.

Through hydraulic fracturing simulations of the reservoir, the geometrical characteristics of fractures at all hierarchical levels can be obtained. These include quantifiable parameters such as individual fracture length and height, enabling the classification of fracture types and facilitating the accurate determination of the total fluid-loss surface area.

By applying the aforementioned methodology, the volume of fracturing fluid loss observed in core fluid-invasion damage experiments can be quantitatively estimated.

In a preferred embodiment, the fluid-loss depth for each fracture type, including the primary fracture and all associated branch fractures, can be individually calculated, yielding:
Fluid-loss depth in the primary hydraulic fracture: D_{loss-fmain}, cm;
Fluid-loss depth in branch fracture 1: D_{loss-fbranch1}, cm;
Fluid-loss depth in branch fracture 2: D_{loss-fbranch2}, cm;
Fluid-loss depth in branch fracture n: D_{loss-fbranchn}, cm.

Additionally, the matrix permeability adjacent to the fracture surfaces can be separately calculated for the primary fracture and each branch fracture, resulting in:
Matrix permeability adjacent to the surface of the primary fracture: K_{m-fmain}, 10⁻³ μm²
Matrix permeability adjacent to the surface of branch fracture 1: K_{m-fbranch1}, 10⁻³ μm²
Matrix permeability adjacent to the surface of branch fracture 2: K_{m-fbranch2}, 10⁻³ μm²
Matrix permeability adjacent to the surface of branch fracture n: K_{m-fbranchn}, 10⁻³ μm²

Accordingly, the total volume of fracturing fluid loss into the formation can be expressed as:

$${V}_{l o s s} = ( {D}_{l o ss - fmain} {H}_{fmain} {L}_{fmain} {e}^{( \frac{1}{b} ) l n ( {K}_{m - fmain} / a )} + {D}_{l oss - fbranch 1} {H}_{fbranch 1} {L}_{fbranch 1} {e}^{( \frac{1}{b} ) l n ( {K}_{m - fbranch 1} / a )} + {D}_{loss - fbranch 2} {H}_{fbranch 2} {L}_{fbranch 2} {e}^{( \frac{1}{b} ) l n ( {K}_{m - fbranch 2} / a )} + … {D}_{loss - fbranchn} {H}_{f b r a n c h n} {L}_{fbranchn} {e}^{( \frac{1}{b} ) l n ( {K}_{m - fbranchn} / a )} ) \times 1 {0}^{- 2}$$

Accordingly, a more accurate calculation result for the total fracturing fluid loss can be obtained.

In a specific embodiment, a core damage test was conducted on Well A-1, a vertical well in a western oilfield reservoir, using the ZF-1 fracturing fluid system (with a fluid viscosity μ_{fluid}=30 mPa·s and fluid density ρ_{fluid}=1.08 g/cm3. Simulated formation water with identical salinity to the reservoir formation water was used to determine the matrix permeability of the reservoir, yielding a value of K_{rock}=20×10⁻³ μm².

Based on downhole core testing conducted in Well A-1, under inlet pressure P_{fracture}=60 MPa and outlet pressure P_{formation}=45 MPa, the measured fracturing fluid damage rate of the ZF-1 fluid system was η_{damage}=0.45, with a resulting damage length L_{damage} 8 cm within the core.

Furthermore, the regression coefficients for the reservoir matrix porosity-permeability relationship were calculated as a=0.0016, b=3.3205. The main hydraulic fracture was 120 meters in length and 30 meters in height, while each of the four branch fractures had a fracture length of 40 meters and a height of 30 meters.

Therefore, the parameter representing the variation of the reservoir matrix permeability to fracturing fluid with respect to penetration depth is:

$${K}_{m} ( i ) = \{ \begin{matrix}20 ( 0.55 + 0.45 \frac{tansig ( i )}{tansig ( 8 )} ) & ( i \leq 8 ) \\ 20 & ( 8 \leq i \leq {D}_{loss}\end{matrix}$$

Accordingly, the calculation formula for the reservoir fracturing fluid loss depth is further transformed into the following expression:

$${P}_{formation ( i )} = {P}_{fracture} - \frac{7 {μ}_{fluid}^{2} {e}^{( \frac{1.5}{b} ) l n ( {K}_{rock} / a )} {D}_{loss ( i )}}{2 {p}_{fluid} {{K}_{m} ( i )}^{1.5} ( {P}_{fracture} - {P}_{formation} )} \times 1 {0}^{- 2.5}$$

The iterative calculation of the penetration depth i is initiated from i=0, and continues until the formation pressure at depth i, denoted as P_{formation} (i), equals the reservoir pore pressure P_{formation}. The corresponding depth i at which this condition is satisfied is defined as the fracturing fluid loss depth Dₗₒₛₛ. In this case, the calculated Dₗₒₛₛ=1.41 cm.

Based on the average fracture height and fracture length of the primary hydraulic fracture, as well as the average fracture height and fracture length of the branched fractures, the total fracturing fluid loss area can be determined as follows:

$$Af = 8400 {m}^{2}$$

Ultimately, the total fluid loss volume of the fracturing fluid is determined as:

$${V}_{loss} = 20.2 \; 87 {m}^{3}$$

The calculation of fracturing fluid loss in the reservoir matrix provided in the embodiments of this specification takes into account the influence of key factors such as reservoir permeability, fracturing fluid viscosity; fracturing fluid density, reservoir fluid pressure, and fracture fluid pressure. It enables the evaluation of fluid loss-induced damage and allows for rapid and accurate quantification of the fracturing fluid loss, thereby providing a reliable basis for the optimal design of the fracturing fluid volume required in hydraulic fracturing operations. Additionally, this method may be integrated with well logging, core analysis, and other datasets to assess the dominant factors controlling the fracturing fluid loss.

Based on the aforementioned method for calculating fracturing fluid loss in the reservoir matrix, the embodiments of this specification further provide a corresponding device for calculating reservoir matrix fracturing fluid loss. The device may include systems (including distributed systems), software (applications), modules, components, servers, clients, or other apparatuses employing the method described in the embodiments herein, in conjunction with the necessary implementation hardware. In accordance with the same inventive concept, one or more embodiments of the apparatus described in this specification are detailed as follows. Since the implementation of the apparatus to address the problem is similar to the method, the detailed implementation of the apparatus may refer to the foregoing description of the method and will not be repeated herein. The terms “unit” or “module” used below may refer to combinations of software and/or hardware configured to perform a predetermined function. Although the apparatuses described in the following embodiments are preferably implemented in software, implementations in hardware, or a combination of hardware and software, are also possible and contemplated.

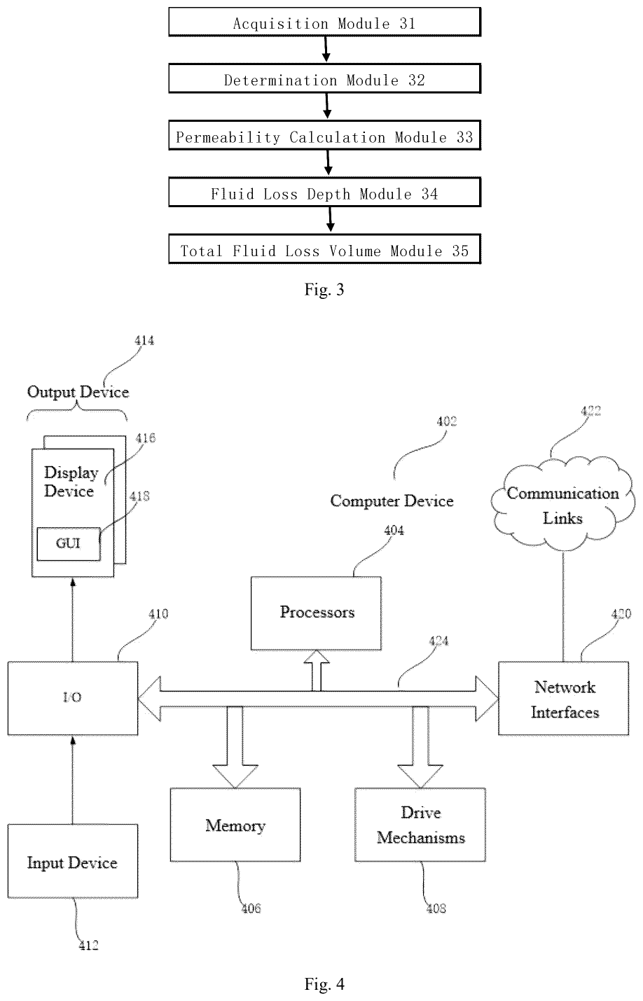

As shown in FIG. 3, the reservoir matrix fracturing fluid loss calculation device according to an embodiment of this specification may comprise:
Acquisition module 31, configured to acquire the pressure data from each measurement point of the core during the fracturing fluid damage experiment of the core;
Determination module 32, configured to determine the fracturing fluid damage length in the core based on the acquired pressure data;
Permeability calculation module 33, configured to calculate the variation parameters of the reservoir matrix permeability to fracturing fluid with respect to penetration depth, based on the determined fracturing fluid damage length;
Fluid loss depth calculation module 34, configured to calculate the reservoir fracturing fluid loss depth based on the fracturing fluid density, fracturing fluid viscosity, and the variation parameters of the reservoir matrix permeability to fracturing fluid with respect to penetration depth, wherein the calculated reservoir fracturing fluid loss depth is:

$${D}_{l o s s} = \frac{90.35 \times {p}_{fluid} {{K}_{m} ( i )}^{1.5} ( {P}_{fracture} - {P}_{formation} )}{{μ}_{fluid}^{2} {e}^{( \frac{1.5}{b} ) l n ( {K}_{rock} / a )}}$$

In which: Dₗₒₛₛ is the fracturing fluid loss depth, cm; ρ_{fluid} is the density of the fracturing fluid, g/cm³; Kₘ(i) is the permeability at a position corresponding to a penetration depth of i, 10⁻³ μm²; i is the penetration depth of the fracturing fluid into the reservoir matrix, cm; P_{fracture} is the fluid pressure inside the fracture, MPa; P_{formation} is the reservoir pore pressure, MPa; μ_{fluid} is the viscosity of the fracturing fluid, mPa·s; K_{rock} is the intrinsic permeability of the reservoir matrix, 10⁻³ μm²; a, b are the dimensionless regression coefficients of the porosity-permeability relationship for the reservoir matrix.

Total Fluid Loss Calculation Module 35, configured to calculate the total fluid loss volume of the fracturing fluid based on the average fracture height and fracture length of the primary hydraulic fracture, the average fracture height and fracture length of the branch fractures, and the calculated fracturing fluid loss depth within the reservoir.

The beneficial effects achieved by the apparatus provided in the embodiments of this specification are consistent with those achieved by the above-described method and will not be repeated here.

As shown in FIG. 4, a computer device provided in an embodiment of this specification is illustrated. The reservoir matrix fracturing fluid loss calculation apparatus described in this specification may be implemented by the computer device in the present embodiment to execute the above-mentioned method. The computer device 402 may comprise one or more processors 404, such as one or more Central Processing Units (CPUs), each of which may implement one or more hardware threads. The computer device 402 may further include any memory 406 for storing any kind of information such as code, settings, and data. Non-limiting examples include any of the following alone or in combination: any type of RAM, any type of ROM, flash memory devices, hard drives, optical disks, etc. More generally, any memory may use any technology for storing information. Moreover, any memory may provide either volatile or non-volatile retention and may represent either a fixed or removable component of the computer device 402. In one scenario, when the processor 404 executes instructions stored in any memory or combination of memories, the computer device 402 may perform operations associated with such instructions. The computer device 402 may also include one or more drive mechanisms 408 for interacting with memory, such as a hard drive mechanism or optical drive.

The computer device 402 may further comprise an input/output (I/O) module 410, which is configured to receive various inputs (via input device 412) and provide various outputs (via output device 414). A specific output mechanism may include a display device 416 and an associated graphical user interface (GUI) 418. In other embodiments, the I/O module 410, input device 412, and output device 414 may be omitted, and the system may operate solely as a networked computing device. The computer device 402 may also include one or more network interfaces 420, configured to exchange data with other devices via one or more communication links 422. One or more communication buses 424 couple the components described above.

The communication links 422 may be implemented in any manner, including local area networks (LAN), wide area networks (WAN, e.g., the Internet), point-to-point links, or any combination thereof. The communication links 422 may involve any combination of hardwired or wireless connections, protocols, routers, gateways, name servers, etc.

Corresponding to the methods shown in FIGS. 1 through 2, the present specification also provides a computer-readable storage medium storing a computer program, which, when executed by a processor, performs the steps of the aforementioned method.

The present specification further provides computer-readable instructions which, when executed by a processor, cause the processor to perform the method as shown in FIGS. 1 through 2.

The present specification also discloses a computer program product, comprising at least one instruction or at least one program segment, which is loaded and executed by a processor to implement the method steps as illustrated in FIGS. 1 through 2.

It should be understood that in various embodiments of this specification, the numbering sequence of the steps does not imply a required order of execution. The actual execution order of the steps should be determined based on their functional logic, and should not be construed as limiting the implementation process of the described embodiments.

It should also be noted that in this specification, the term “and/or” is merely used to describe an inclusive relationship between associated elements, encompassing three scenarios: A alone, B alone, or both A and B. Additionally, the symbol “/” generally indicates a logical “or” relationship between the preceding and following elements.

A person of ordinary skill in the art will understand that the units and algorithm steps described in connection with the disclosed embodiments can be implemented in hardware, computer software, or a combination thereof. To clearly illustrate the interchangeability of hardware and software, the components and steps have been described functionally in general terms. Whether these functions are implemented in hardware or software depends on the specific application and design constraints. A person skilled in the art may implement the described functions using different approaches, but such implementations should not be construed as beyond the scope of this specification.

Those skilled in the art will clearly recognize that, for the sake of convenience and brevity, the detailed working processes of the described systems, apparatuses, and units can refer to the corresponding methods provided in the method embodiments and will not be repeated here.

In the embodiments provided in this specification, it should be understood that the disclosed systems, devices, and methods can also be implemented in other ways. For example, the device embodiments described above are merely illustrative. The functional units may be logically divided in various ways; multiple units or components may be combined or integrated into another system, certain features may be omitted or not executed. Additionally, the described connections or communications may be direct or indirect, or realized via interfaces or intermediary units, and may involve electrical, mechanical, or other forms of connection.

The described units, although illustrated as separate components, may or may not be physically separate. They may be localized or distributed across multiple network nodes. The practical implementation may involve all or part of these units depending on the application objectives.

Furthermore, in the embodiments described in this specification, functional units may be integrated into a single processing unit or may exist separately. They may be implemented using hardware or as software function modules.

If the integrated unit is implemented as a software function module and sold or used as a standalone product, it may be stored on a computer-readable storage medium. Based on this understanding, the technical solutions described herein, or at least part thereof, may be embodied as a software product stored on a storage medium and comprising instructions that enable a computer device (e.g., personal computer, server, network equipment) to execute all or part of the method steps described in various embodiments of this specification. The storage medium may include: USB flash drives, portable hard disks, ROM, RAM, magnetic disks, optical disks, or any other medium capable of storing program code.

The embodiments and examples provided in this specification are intended to illustrate the principles and implementation of the described methods and systems. These descriptions are not intended to limit the scope of the specification. Those skilled in the art, based on the disclosed ideas, may make changes or modifications in specific implementation details and application ranges. Therefore, the content of this specification should not be interpreted as limiting the scope of protection.

1. A method for calculating the leak-off volume of fracturing fluid in a reservoir matrix, characterized in that the method comprises: Acquiring pressure data from each pressure measurement point within the core during the core fracturing fluid damage experiment; Based on the pressure data, the fracturing fluid invasion length of the core is determined; Calculating parameters that characterize the variation of reservoir matrix permeability to fracturing fluid as a function of invasion depth, based on the determined fracturing length; Based on the fracturing fluid density, fracturing fluid viscosity; and the parameter representing the variation of the reservoir matrix permeability to fracturing fluid with respect to penetration depth, the fracturing fluid loss depth in the reservoir is calculated. The resulting fracturing fluid loss depth is:
$${D}_{l o s s} = \frac{90.35 \times {p}_{fluid} {{K}_{m} ( i )}^{1.5} ( {P}_{f racture} - {P}_{f o rmation} )}{{μ}_{fluid}^{2} {e}^{( \frac{1.5}{b} ) l n ( {K}_{rock} / a )}}$$
In which: Dₗₒₛₛ is the fracturing fluid loss depth, cm; ρ_{fluid} is the density of the fracturing fluid, g/cm³; Kₘ(i) is the permeability at a position corresponding to a penetration depth of i, 10⁻³ μm²; i is the penetration depth of the fracturing fluid into the reservoir matrix, cm; P_{fracture} is the fluid pressure inside the fracture, MPa; P_{formation} is the reservoir pore pressure, MPa; μ_{fluid} is the viscosity of the fracturing fluid, mPa·S; K_{rock} is the intrinsic permeability of the reservoir matrix, 10⁻³ μm²; a, b are the dimensionless regression coefficients of the porosity-permeability relationship for the reservoir matrix; Based on the average height and length of the primary hydraulic fracture, the average height and length of the branch fractures, and the fracturing fluid loss depth in the reservoir, the total fracturing fluid loss volume is calculated.

2. The method according to claim 1, characterized in that, based on the pressure data, the fracturing length within the core is determined, further comprising: Obtain the pressure data at each pressure measurement point on the core holder under the conditions of initial inlet pressure, initial outlet pressure, and a predetermined fracturing duration during the fracturing fluid damage test; Based on the pressure data obtained from each pressure measurement point, along with the initial inlet pressure and the initial outlet pressure, the pressure gradient deviation coefficient for each pressure measurement point is calculated; Determine whether any of the pressure measurement points exhibit a pressure gradient differential coefficient that satisfies the predetermined criteria; When a predicted point exists where the pressure gradient differential coefficient satisfies a predefined condition, the fracturing fluid invasion damage length is determined based on the pressure measurement points meeting the predefined condition of the pressure gradient differential coefficient and the number of repetitions of the fracturing fluid damage experiment; When none of the pressure measurement points meet the predetermined criteria, the initial inlet pressure shall be updated, and the fracturing fluid damage test shall be repeated for the preset fracturing duration until a predicted point is identified at which the pressure gradient variation coefficient satisfies the predetermined criteria.

3. The method according to claim 2, wherein the pressure gradient deviation coefficient is calculated according to the following equation:
$${η}_{j} = ( {G}_{P j} - {G}_{rock} ) / {G}_{rock}$$
In which: ηⱼ is the pressure gradient deviation coefficient at the j-th pressure measurement point, dimensionless; G_{Pj} is the pressure gradient at the j-th pressure sensing point, MPa/cm; G_{rock} is the pressure gradient between the outlet and the inlet of the core holder, MPa/cm;
$${G}_{Pj} = \frac{{P}_{j} - {P}_{j - 1}}{Δ L}$$
$${G}_{rock} = \frac{{P}_{i n} - {P}_{out}}{L}$$
In which: Pⱼ is the pressure measured at the j-th pressure monitoring point, MPa; Pⱼ₋₁ the pressure at the (j−1)th pressure measurement point, MPa; ΔL is the core length between two adjacent pressure measurement points, cm; Pᵢₙ is the pressure at the inlet of the core holder, MPa; Pₒᵤₜ is the pressure at the outlet end of the core holder, MPa; L is the length of the core, cm.

4. The method according to claim 3, wherein, when a predicted point exists at which the pressure gradient deviation coefficient satisfies a predetermined condition, the fracturing fluid invasion damage length is determined based on the pressure measurement point(s) satisfying the predetermined condition for the pressure gradient deviation coefficient and the number of repetitions of the fracturing fluid damage experiment:
$${L}_{damage} = nL + j Δ L$$
In which: L_{damage} is the fracturing fluid invasion damage length, cm; n is the number of repetitions of the fracturing fluid damage test; L is the length of the core, cm; ΔL is the core length between two adjacent pressure measurement points, cm; j is the index of j-th the pressure measurement point.

5. The method according to claim 3, wherein, when none of the pressure measurement points satisfy the predefined conditions, the initial inlet pressure is updated, further comprising: In a previous fracturing fluid damage experiment, when the fracturing duration reached the predetermined pressure holding time, the outlet pressure of the core holder was updated to the initial inlet pressure.

6. The method according to claim 1, wherein, based on the fracturing fluid damage length, the variation parameter of the reservoir matrix permeability to fracturing fluid as a function of penetration depth is calculated, further comprising:
$${K}_{m} ( i ) = \{ \begin{matrix}{K}_{rock} ( 1 - {η}_{damage} + {η}_{damage} \frac{tansig ( i )}{tansig ( Ldamage )} ) & ( i \leq {L}_{damage} ) \\ {K}_{rock} & ( {L}_{damage} \leq i \leq {D}_{loss} )\end{matrix}$$
In which: Kₘ(i) is the permeability at a position corresponding to a penetration depth of i, 10⁻³ μm²; K_{rock} is the intrinsic permeability of the reservoir matrix, 10⁻³ μm²; η_{damage} is the fracturing fluid damage coefficient, dimensionless; L_{damage} is the fracturing fluid invasion damage length, cm; tansig is the transfer function, which is specifically calculated by the following expression:
$$tansig ( i ) = \frac{{e}^{i} - {e}^{- i}}{{e}^{i} + {e}^{- i}}$$
i is the penetration depth of the fracturing fluid into the reservoir matrix, cm.

7. The method according to claim 1, wherein the total fluid loss of the fracturing fluid is further calculated based on the average fracture height and fracture length of the primary hydraulic fracture, the average fracture height and fracture length of the branch fractures, and the fracturing fluid loss depth within the reservoir formation, thereby providing a basis for:
$${V}_{loss} = {D}_{loss} {e}^{( \frac{1}{b} ) l n ( {K}_{m} ( i ) / a )} ( {H}_{fmain} {L}_{fmain} + {H}_{fbranch 1} {L}_{fbranch 1} + {H}_{fbranch 2} {L}_{fbranch 2} + {H}_{fbranchn} {L}_{fbranchn} ) \times 1 {0}^{- 2}$$
In which: Vₗₒₛₛ is the total fluid loss volume of the fracturing fluid, m³; Dₗₒₛₛ is the fracturing fluid loss depth, cm; Kₘ(i) is the permeability at a position corresponding to a penetration depth of i, 10 μm²; H_{fmain} is the average fracture height of the primary hydraulic fracture, m; L_{fmain} is the half-length of primary hydraulic fracture, m; H_{fbranch1} is the average fracture height for hydraulic fracture of branch 1, m; L_{fbranch1} is the half-length for hydraulic fracture of branch 1, m; H_{fbranch2} is the average fracture height for hydraulic fracture of branch 2, m; L_{fbranch2} is the half-length for hydraulic fracture of branch 2, m; H_{fbranchn} is the average fracture height for hydraulic fracture of branch n, m; L_{fbranchn} is is the half-length for hydraulic fracture of branch n, m.

8. A reservoir matrix fracturing fluid loss calculation apparatus, characterized by comprising: An acquisition module configured to obtain pressure data from multiple measurement points along the core during the fracturing fluid damage testing of the core; Determination module, configured to determine the fracturing fluid invasion damage length of the core based on the pressure data; Permeability Calculation Module, configured to calculate the variation parameter of the reservoir matrix permeability to fracturing fluid as a function of penetration depth, based on the fracturing fluid invasion damage length; Fluid Loss Depth Calculation Module, configured to determine the fracturing fluid loss depth within the reservoir formation based on the fracturing fluid density, fracturing fluid viscosity, and the depth-dependent permeability variation parameter of the reservoir matrix to the fracturing fluid. The resulting fracturing fluid loss depth is:
$${D}_{loss} = \frac{90.35 \times {p}_{fluid} {{K}_{m} ( i )}^{1.5} ( {P}_{f racture} - {P}_{formation} )}{{μ}_{fluid}^{2} {e}^{( \frac{1.5}{b} ) l n ( {K}_{r o c k} / a )}}$$
In which: Dₗₒₛₛ is the fracturing fluid loss depth, cm; ρ_{fluid} is the density of the fracturing fluid, g/cm³; Kₘ(i) is the permeability at a position corresponding to a penetration depth of i, 10⁻³ μm²; i is the penetration depth of the fracturing fluid into the reservoir matrix, cm; P_{fracture} is the fluid pressure inside the fracture, MPa; P_{formation} is the reservoir pore pressure, MPa; μ_{fluid} is the viscosity of the fracturing fluid, mPa·s; K_{rock} is the intrinsic permeability of the reservoir matrix, 10⁻³ μm²; a, b are the dimensionless regression coefficients of the porosity-permeability relationship for the reservoir matrix; Total fluid loss calculation module: configured to calculate the total fluid loss of the fracturing fluid based on the average fracture height and fracture length of the primary hydraulic fracture, the average fracture height and fracture length of the branch fractures, and the fracturing fluid loss depth within the reservoir.

9. A computing device comprising a memory, a processor, and a computer program stored on the memory and executable by the processor, characterized in that, when executing the computer program, the processor is configured to implement the method according to any one of claims 1 to 7.
